(19) United States
(12) Patent Application Publication
(10) Pub. No.: US 2025/0026709 A1
(43) Pub. Date: Jan. 23, 2025

(54) PROCESS FOR ALKOXYCARBONYLATION OF DIISOBUTENE AND A C4 TO C7 OLEFIN

(71) Applicant: Evonik Oxeno GmbH & Co. KG, Marl (DE)

(72) Inventors: Peter KUCMIERCZYK, Herne (DE); Maren TERMÜHLEN, Dortmund (DE); Annika REINHARDT, Marl (DE); Armin Matthias RIX, Marl (DE); Robert FRANKE, Marl (DE); Dirk FRIDAG, Haltern am See (DE); Anna Chiara SALE, Düsseldorf (DE); Ana MARKOVIC, Haltern am See (DE)

(73) Assignee: Evonik Oxeno GmbH & Co. KG, Marl (DE)

(21) Appl. No.: 18/776,364

(22) Filed: Jul. 18, 2024

(30) Foreign Application Priority Data
Jul. 20, 2023 (EP) 23186733.4

(51) Int. Cl. C07C 67/38; B01J 23/44

(52) U.S. Cl. CPC C07C 67/38; B01J 23/44

(57) ABSTRACT

The invention provides a process for alkoxycarbonylation of disobutone and a C4 to C7 olefin in a common reaction zone. The alkoxycarbonylation is carried out with an alcohol and carbon monoxide in the presence of a homogeneous catalyst system that comprises at least one metal from group 8 to 10 of the periodic table of the elements or a compound thereof, a phosphorus-containing ligand and an acid.

The present invention relates to a process for alkoxycarbonylation of diisobutene and a C4 to C7 olefin in a common reaction zone. The alkoxycarbonylation is carried out with an alcohol and carbon monoxide in the presence of a homogeneous catalyst system that comprises at least one metal from group 8 to 10 of the periodic table of the elements or a compound thereof, a phosphorus-containing ligand and an acid.

Diisobutene is an industrially relevant product obtained by dimerization of isobutene. Diisobutene consists of the isomers 2,4,4-trimethylpent-1-ene (hereinbelow also: TMP1) and 2,4,4-trimethylpent-2-ene (hereinbelow also: TMP2) with a mass distribution of TMP1:TMP2 in the range of about 78:22 to 81:19 (equilibrium distribution). Industrial mixtures comprising C4 olefins are light petroleum fractions from refineries, C4 fractions from FC crackers or steam crackers, mixtures from Fischer-Tropsch syntheses, mixtures from dehydrogenation of butanes, and mixtures resulting from metathesis or other industrial processes. C5 olefins, i.e. pentenes, are present in light petroleum fractions from refineries or crackers. The higher olefins are obtainable in particular by oligomerization reactions. Both diisobutene and C4 to C7 olefins may be converted by alkoxycarbonylation into valuable products such as the esters formed in the alkoxycarbonylation.

The problem with such processes is that dedicated production plants must be available or constructed and that these plants must be operated with the costs that this entails. Since the markets for petrochemical products are in some cases rather variable, it is hardly possible to operate dedicated production plants for each of the recited olefins economically. A further disadvantage is that resource-efficient operation of several production plants is hardly possible because all plants require maintenance. This entails not only economic and personnel costs but also necessitates certain amounts of energy such as electricity or heat transfer medium.

It is accordingly an object of the present invention to provide a process which does not exhibit the aforementioned problems. It should especially be possible to convert both diisobutene and C4 to C7 olefins into valuable products via alkoxycarbonylation in a more resource-efficient manner.

The underlying object was achieved by the process described in claim 1. Preferred embodiments are specified in the dependent claims.

According to the invention the process for alkoxycarbonylation of diisobutene and a C4 to C7 olefin comprises at least the following steps:
a. providing a diisobutene stream containing 2,4,4-trimethylpent-2-ene and 2,4,4-trimethylpent-1-ene and providing an olefin stream containing the C4 to C7 olefin, preferably C4 olefins;
b. alkoxycarbonylation of the diisobutene and the C4 to C7 olefin, preferably C4 olefin with an alcohol and carbon monoxide in the presence of a homogeneous catalyst system comprising at least one metal of group 8 to 10 of the periodic table of the elements or a compound thereof, a phosphorus-containing ligand and an acid in a common reaction zone to obtain a liquid product mixture comprising at least the esters formed by the alkoxycarbonylation, the homogeneous catalyst system, unreacted olefins, i.e. unreacted diisobutene and unreacted C4 to C7 olefins, and unreacted alcohol;
c. removing the homogeneous catalyst system from the liquid product mixture to obtain a crude product mixture comprising at least the esters formed by the alkoxycarbonylation, the unreacted olefins and unreacted alcohols; and
d. distillative processing of the crude product mixture in at least one distillation column to remove the unreacted alcohols and the unreacted olefins to obtain an ester mixture containing the esters formed, wherein preferably unreacted alcohol and unreacted olefins are removed and recycled to the alkoxycarbonylation in step b.

The process according to the invention therefore relates to the simultaneous reaction of diisobutene and C4 to C7 olefins in a single common reaction zone. Such a procedure has numerous advantages.

The process described makes it possible to react flexibly to markets, in particular small production amounts. In addition, only one production plant is necessary and said plant can also be operated more efficiently and thus in a more resource-saving manner even when market requirements fluctuate. The specific boiling order also makes it possible to separate the obtained products of the respectively employed olefins, and the solvent methanol, while the reactants may be returned to the reaction directly or after additional processing.

The diisobutene stream provided in step a comprises 2,4,4-trimethylpent-2-ene and 2,4,4-trimethylpent-1-ene. In a preferred embodiment, the proportion of 2,4,4-trimethylpent-1-ene in the diisobutene stream is at least 60 mol %, preferably at least 70 mol %, based on the total diisobutene stream. Such streams may be diisobutene streams produced by dimerization from isobutene or isobutene-containing hydrocarbon mixtures, for example according to the process disclosed in EP 1 360 160 B1. In addition, the diisobutene streams to be employed here may be obtained as unreacted residual streams in carbonylation processes, for example in alkoxycarbonylation or in hydroformylation.

Step a comprises providing not only the diisobutene stream but also an olefin stream which contains the C4 to C7 olefin employed in the process according to the invention. In a preferred embodiment the present invention employs an olefin stream comprising C4 olefins, particularly preferably a C4 olefin stream. Corresponding streams are known to those skilled in the art and available on a large industrial scale.

Olefin streams comprising C4 olefins include for example light petroleum fractions from refineries, C4 fractions from FC crackers or steam crackers, mixtures from Fischer-Tropsch syntheses, mixtures from dehydrogenation of butanes or streams resulting from metathesis or from other industrial processes. For example, C4 olefin streams suitable for the process according to the invention are obtainable from the C4 fraction of a steam cracker. C5 olefins, i.e. pentenes, are present in light petroleum fractions from refineries or crackers. C6 olefins are obtainable for example by dimerization of propene. C7 olefins are obtainable for example by dimerization of propylene and butene.

The streams provided in step a, i.e. the diisobutene stream and the C4 to C7 olefin stream, are passed to the alkoxycarbonylation in step b. The streams may be individually and separately sent to the alkoxycarbonylation in step b or mixed beforehand. The diisobutene stream and the C4 to C7 olefin stream are preferably mixed prior to the alkoxycarbonylation in step b. A particularly preferred embodiment of the present invention in fact provides that the diisobutene stream, the C4 to C7 olefin stream, the alcohol and the homogeneous catalyst system are mixed, in particular in a suitable mixing vessel, prior to the alkoxycarbonylation in step b. If there is a recycle stream to the reaction, i.e. by recycling the catalyst system for example, this recycle stream can likewise be passed to the mixing vessel.

Step b comprises reacting the diisobutenes, i.e. 2,4,4-trimethylpent-2-ene and 2,4,4-trimethylpent-1-ene, and the C4 to C7 olefin with carbon monoxide (CO) and an alcohol to afford an ester. The number of carbon atoms in the ester thus increases, compared to the olefin used, by 1 carbon atom from the carbon monoxide and by the number of carbon atoms in the alcohol used. The diisobutenes (8 carbon atoms) accordingly give rise to an ester having 9 carbon atoms in the acid part of the ester, plus the carbon atoms of the alcohol in the alcohol part of the ester.

In the alkoxycarbonylation in step b the carbon monoxide may be provided directly in the form of an input mixture or through addition of a carbon monoxide-containing gas selected from synthesis gas, water gas, generator gas and other carbon monoxide-containing gases. It is also possible to provide the carbon monoxide by first separating the carbon monoxide-containing gas into its components in a manner known to those skilled in the art and passing the carbon monoxide to the reaction zone. The carbon monoxide may still contain a certain proportion of hydrogen or other gases, because complete separation is almost impossible.

The alcohol used in the reaction in step b is preferably a monohydric alcohol having 1 to 4 carbon atoms, especially methanol, ethanol, propanol or butanol. This then affords the esters with a chain length corresponding to the employed alcohol. For example the use of methanol results in formation of the methyl ester.

The alcohol employed in step b, preferably the monohydric alcohol having 1 to 4 carbon atoms, in particular methanol, ethanol, propanol or butanol, is employed in a molar ratio to the total amount of all olefins, i.e. the diisobutenes and the C4 to C7 olefins, alcohol:olefin, in a range from 10:1 to 1:1, preferably 8:1 to 1.5:1, particularly preferably 7:1 to 2:1. The alcohol is thus added at least in an identical molar amount based on the employed olefins, but preferably in a molar excess.

The reaction according to the invention in step b is performed in the presence of a homogeneous catalyst system that comprises at least one metal from group 8 to 10 of the periodic table of the elements (PTE) or a compound thereof, a phosphorus-containing ligand and an acid as co-catalyst. It goes without saying that a suitable catalyst must be capable of alkoxycarbonylating all olefins, i.e. the diisobutenes and the C4 to C7 olefins. The content of the metal of group 8 to 10 of the periodic table of the elements, in particular of palladium, in the alkoxycarbonylation reaction solution in step b is preferably 100 to 500 ppm, further preferably 150 to 450 ppm, particularly preferably 180 to 350 ppm.

The metal from group 8 to 10 of the PTE is preferably palladium. The palladium is preferably used in the form of a precursor compound as a palladium compound coordinated by the phosphorus-containing ligand. Examples of palladium compounds that may be used as precursor compounds are palladium chloride [PdCl₂], palladium (II) acetylacetonate [Pd(acac)₂], palladium(II) acetate [Pd(OAc)₂], dichloro(1,5-cyclooctadiene)palladium(II) [Pd(cod)₂Cl2], bis(dibenzylideneacetone)palladium(0) [Pd(dba)₂], tris(dibenzylideneacetone)dipalladium(0) [Pd₂(dba)₃] bis(acetonitrile)dichloropalladium(II) [Pd(CH₃CN)₂Cl₂], palladium(cinnamyl)dichloride [Pd(cinnamyl)Cl₂]. Preference is given to using the compounds [Pd(acac)₂] or [Pd(OAc)₂]. The concentration of palladium metal in step b is preferably between 0.01 and 0.6 mol %, preferably between 0.03 and 0.3 mol %, more preferably between 0.04 and 0.2 mol %, based on the molar amount of the hydrocarbon used.

Suitable phosphorus-containing ligands of the catalyst system according to the invention preferably have a bidentate structure. Preferred phosphorus-containing ligands for the catalyst system according to the invention are benzene-based diphosphine compounds, as disclosed, for example, in EP 3 121 184 A2. The ligands may be combined with the palladium in a preliminary reaction so that the palladium-ligand complex is passed to the reaction zone, or added to the reaction in situ and combined with the palladium there. The molar ratio of ligand:metal for the described reaction in step b 1 may be: 1 to 10:1, preferably 2:1 to 6:1, particularly preferably 3:1 to 5:1.

The homogeneous catalyst system further comprises an acid, in particular a Brønsted or a Lewis acid. Lewis acids used are preferably aluminium triflate, aluminium chloride, aluminium hydride, trimethylaluminium, tris(pentafluorophenyl) borane, boron trifluoride, boron trichloride or mixtures thereof. Of the Lewis acids mentioned, preference is given to using aluminium triflate. The Lewis acid is preferably added in a molar ratio of Lewis acid:ligand of 1:1 to 20:1, preferably 2:1 to 15:1, particularly preferably 5:1 to 10:1.

Suitable Brønsted acids preferably have an acid strength of pKa≤5, more preferably an acid strength of pKa≤3. The stated acid strength pKa relates to the pKa determined under standard conditions (25° C., 1.01325 bar). For a polyprotic acid, the acid strength pKa relates in the context of this invention to the pKa of the first protolysis step. The Brønsted acid is preferably added in a molar ratio of Brønsted acid:ligand of 1:1 to 15:1, preferably 2:1 to 10:1, particularly preferably 3:1 to 5:1.

The Brønsted acid used may in particular be perchloric acid, sulfuric acid, phosphoric acid, methylphosphonic acid or sulfonic acids. Suitable sulfonic acids are for example methanesulfonic acid, trifluoromethanesulfonic acid, tert-butanesulfonic acid, p-toluenesulfonic acid (PTSA), 2-hydroxypropane-2-sulfonic acid, 2,4,6-trimethylbenzenesulfonic acid and dodecyl sulfonic acid. Particularly preferred acids are sulfuric acid, methanesulfonic acid, trifluoromethanesulfonic acid and p-toluenesulfonic acid. The acid is preferably sulfuric acid.

The alkoxycarbonylation in step b is preferably carried out at a temperature in the range from 60° C. to 120° C., further preferably in the range from 65° C. to 110° C., particularly preferably in the range from 70° C. to 100° C. The alkoxycarbonylation in step b is further preferably carried out at a carbon monoxide pressure of from 10 to 35 bar, preferably 12.5 to 30 bar, more preferably 15 to 25 bar.

The alkoxycarbonylation in step b takes place in a suitable reaction zone. The reaction zone for the reaction comprises at least one reactor, but may also consist of two or more reactors arranged in parallel or in series. The at least one reactor may in particular be selected from the group consisting of a stirred-tank reactor, a loop reactor, a jet-loop reactor, a bubble-column reactor or combinations thereof. If more than one reactor is used, the reactors may be identical or different.

The alkoxycarbonylation in step b described above affords a liquid product mixture that comprises at least the esters formed by the alkoxycarbonylation, the homogeneous catalyst system, unreacted olefins, and unreacted alcohol. In addition, the liquid product mixture may contain low-boiling by-products such as dimethyl ether or formic acid and/or high-boiling components such as ligand decomposition products.

The product mixture thus obtained is supplied to the following step c in order to remove the homogeneous catalyst system from the liquid product mixture. The supplying of the product mixture may already be preceded by removal of low-boiling components, for example a portion of the unreacted alcohol and/or of the low-boiling by-products, for example by distillation. The alcohol can be recycled to step b, i.e. to the alkoxycarbonylation. The low-boiling by-products can be discharged from the process in the context of a purge, so that they do not accumulate in the process. The removal of the homogeneous catalyst system affords a crude product mixture that comprises at least the esters formed by the alkoxycarbonylation, unreacted olefins and at least a portion of the unreacted alcohols.

The removal of the homogeneous catalyst system to obtain the crude product mixture in step c may be effected with the aid of various separation processes, for example by thermal separation or by membrane separation. Suitable processes are familiar to those skilled in the art. The removal of the homogeneous catalyst system in step c is in the context of the present invention preferably effected by membrane separation. As is known, a membrane separation gives rise to a retentate and a permeate. The homogeneous catalyst system will accumulate in the retentate. The permeate in that case constitutes the crude product mixture mentioned previously and is sent to subsequent step d of distillative processing.

It is preferable in accordance with the invention when the retentate is recycled to the alkoxycarbonylation in step b/to the reaction zone where the alkoxycarbonylation is carried out. This allows the catalyst system to be reused. In the preferably continuous execution of the claimed process, this gives rise to a catalyst cycle in which at most only minor process-related catalyst losses need to be compensated. If according to the preferred embodiment the diisobutene stream, the olefin stream, the alcohol and the homogeneous catalyst system are mixed especially in a suitable mixing vessel prior to the alkoxycarbonylation in step b, the retentate is sent to the mixing vessel. In the recycling of the retentate, a purge stream that may contain inert alkanes, low-boiling by-products (for example ethers), possible decomposition products of the catalyst system or other impurities, for example traces of water or nitrogen, may additionally be withdrawn to avoid accumulation in the reaction zone(s).

Any suitable membrane material may be used for the membrane separation. Preference is given to using an OSN (organic solvent nanofiltration) membrane material in the membrane separation in step c of the process of the invention. Such a membrane material preferably consists at least of a separation-active layer (also: active separation layer) and a substructure on which the separation-active layer is present. The membrane material of the invention preferably consists at least of a separation-active layer and a substructure.

The membrane material, composed at least of separation-active layer and a substructure, should be acid-stable so that the membrane material is not damaged by the acid present in the liquid product mixture. The term “acid stable” means in the context of the present invention that the membrane material is stable for at least 300 h in the presence of the acid of the catalyst system and the separation performance is maintained.

The substructure preferably has a porous structure that is permeable to the permeate that has passed through the separation-active layer. The substructure has a stabilizing function and serves as a support for the separation-active layer. The substructure may in principle consist of any suitable porous material. Suitable materials are familiar to those skilled in the art. A prerequisite, however, is that the material is stable to acids and bases. The substructure may consist of the same material as the separation-active layer. Preferred materials for the substructure include plastics such as polypropylene (PP), polyethylene (PE) or non-condensation polymers that are not susceptible to hydrolytic or alcoholytic cleavage, such as polysulfones, polytetrafluoroethylene (PTFE), polyethersulfone (PES), polyvinylidene fluoride (PVDF) or polyacrylonitrile (PAN).

The separation-active layer according to the invention is preferably composed of a PAEK (polyaryletherketone) polymer. PAEK has the particular feature that, within the repeat unit, aryl groups are linked alternately via an ether functionality and a ketone functionality. A separation-active layer that is preferred according to the invention is composed of PEEK (polyether ether ketone). As the separation-active layer, particular preference is given to using PEEK polymers having a degree of sulfonation of less than 20%, particularly preferably having a degree of sulfonation of less than 10%. The corresponding PEEK polymers and the production thereof are described in WO 2015/110843 A1.

The membrane separation in step c is preferably carried out at a temperature in the range from 25° C. to 100° C., further preferably in the range from 30° C. to 80° C. and particularly preferably in the range from 40° C. to 70° C. To bring the product mixture to the prevailing temperature preferred for the membrane separation, the product mixture may be cooled. In addition to active cooling using a coolant, cooling may also be achieved via a heat exchanger where heat energy is transferred to another stream, thus cooling the product mixture and heating the other stream.

The transmembrane pressure (TMP) in the membrane separation in step c is preferably in the range from 10 to 60 bar, further preferably in the range from 15 to 55 bar, particularly preferably in the range from 20 to 50 bar. The permeate-side pressure here may be above atmospheric pressure and preferably up to 15 bar, preferably 3 to 7 bar. The difference between the TMP and the permeate-side pressure gives the retentate-side pressure. In a preferred embodiment, care should be taken, in the case of the pressure ratios and the permeate-side pressure in particular, to ensure that the pressure is set in accordance with the hydrocarbon used, the alcohol used and the temperature in the system, such that evaporation after passage through the membrane is avoided. Evaporation could lead to unstable operation.

In the subsequent step d, the distillative processing of the crude product mixture/the permeate from the membrane separation is carried out in at least one distillation column to remove the unreacted alcohols and the unreacted olefins, i.e. unreacted diisobutenes and unreacted C4 to C7 olefins. This affords an ester mixture comprising the esters formed. The ester mixture thus contains the esters formed from the diisobutene and the C4 to C7 olefin.

In the distillative processing of the crude product mixture/the permeate in step d the unreacted alcohol and the unreacted olefins, i.e. unreacted diisobutenes and unreacted C4 to C7 olefins, are obtained at the top of the at least one distillation column. The mixture of the esters formed thus accumulates in the bottom of the at least one distillation column. The tops stream comprising the unreacted alcohol removed in the at least one distillation column and the unreacted olefins may be recycled to the alkoxycarbonylation in step b/to the reaction zone. If the components used undergo mixing prior to the alkoxycarbonylation, the overhead stream is by definition supplied to the mixing. This allows continuous operation of the process of the invention at the highest possible yield. A purge can be withdrawn from the recycled overhead stream in order to discharge low-boiling by-products from the process.

The distillative processing to remove the unreacted alcohols and the unreacted olefins in step d may be carried out in a distillation column. It would be conceivable for the distillative processing to remove the unreacted alcohols and the unreacted olefins in step d to be carried out in two or more distillation columns. However, this would entail markedly higher apparatus costs. It is therefore preferable that the distillative processing in step d is carried out in a single distillation column.

The pressure in the distillation column in the distillative processing in step d is preferably in the range from 0.3 to 2 bar, more preferably in the range from 0.4 to 1 bar, particularly preferably in the range 0.5 to 0.7 bar. The temperature in the bottom of the distillation column in the distillative processing in step d is preferably in the range from 80° C. to 160° C. The temperature at the top of the distillation column in the distillative processing in step d is preferably in the range from 30° C. to 80° C. In addition, it is preferable when the reflux ratio in the distillation column is between 1 and 2. The distillation column for the removal in step d preferably comprises 10 to 30 theoretical plates. The distillation column may contain high-performance structured packings. Suitable high-performance structured packings are known to those skilled in the art.

As mentioned above the distillative processing affords an ester mixture comprising the esters formed from the diisobutene and the C4 to C7 olefin. To obtain both esters in the highest possible purity a further distillation step may be performed to separate the esters of the ester mixture from one another. The esters from the C4 to C7 olefin will be obtained at the top of the distillation column and the esters from the diisobutene at the bottom of the distillation column.

The present process is suitable for alkoxycarbonylation of diisobutene and C4 olefins with an alcohol, preferably an alcohol having 1 to 4 carbon atoms. Certain combinations of olefins and alcohols are particularly preferred in the context of the present invention:

In a preferred embodiment, the process relates to the alkoxycarbonylation of diisobutene and a C4 olefin, i.e. 1-butene, cis- and/or trans-2-butene, isobutene or mixtures thereof, with methanol. The ester formed from diisobutene in the alkoxycarbonylation with methanol is methyl 3,5,5-trimethylhexanoate. The alkoxycarbonylation with methanol of the different butenes can form methyl valerate, methyl 2-methylbutyrate and/or methyl 3-methylbutyrate. Employing a mixture of butenes accordingly also affords a mixture of the recited esters.

In a preferred embodiment, the process relates to the alkoxycarbonylation of diisobutene and a C4 olefin, i.e. 1-butene, cis- and/or trans-2-butene, isobutene or mixtures thereof, with ethanol. The ester formed from diisobutene in the alkoxycarbonylation with ethanol is ethyl 3,5,5-trimethylhexanoate. The alkoxycarbonylation with ethanol of the different butenes can form ethyl valerate, ethyl 2-methylbutyrate and/or ethyl 3-methylbutyrate. Employing a mixture of butenes accordingly also affords a mixture of the recited esters.

The present invention is explained hereinbelow by reference to examples. The examples relate to preferred embodiments but are not to be understood as limiting the invention.

### EXAMPLES

### Example 1—Reaction of Diisobutene and 1-Butene

The present experiment employs the substrates diisobutene, here a mixture of 4,4-trimethylpent-1-ene (TMP1) and 2,4,4-trimethylpent-2-ene (TMP2) in a molar ratio of 79:21 (TMP1:TMP2), and 1-butene. The alcohol employed is methanol. The catalyst system employed in the alkoxycarbonylation comprises the following substances: The acid employed was aluminum trifluoromethanesulfonic acid (Al(OTf)3). The ligand employed was 1,2-bis((tert-butyl(pyridin-2-yl)phosphanyl)methyl)benzene (L1) and the precursor for the metal employed is palladium (II) bis(acetylacetonate) (Pd(acac)2). The internal standard used for later quantification is ethylbenzene.

A catalyst stock solution with methanol as solvent and a target content of palladium of about 250 ppm is initially made up. The ligand:Pd ratio (molar) is 4:1 and the Al(OTf)3:ligand ratio (molar) is 5:1. This solution is stirred for 30 minutes at room temperature under an argon atmosphere. Subsequently a portion of this solution is transferred into a reactor. Diisobutene is additionally added. After sealing the reactor the contents are alternately (3 times) pressurized with argon to up to 10 bar and decompressed again to expel oxygen. A pressure of 3 bar of argon is then applied and the reaction solution heated to a temperature of 80° C. with stirring. A vessel filled with 1-butene previously connected to the reactor is connected at its top to a valve which allows introduction of CO gas. The contents of the vessel and CO are transferable into the reaction vessel via the bottom of the vessel by operating the valve.

A pressure of 30 bar of CO is initially established in the vessel. To commence reaction the valve is opened, thus allowing 1-butene and CO into the reaction vessel. The CO is continuously metered in to achieve a constant pressure of 20 bar during the reaction. The progress of the reaction is monitored by sampling. The samples are diluted with isopropanol and measured by gas chromatography. A preceding calibration of the relevant components allows quantification by internal standard ethylbenzene. The results are reported in table 1:

TABLE 1
Progress of reaction according to example 1

| | | | | Yield of | |
|---|---|---|---|---|---|
| | Yield of methyl | Yield of n- | Yield of methyl | C5 | |
| | 3,5,5- | methyl | 2- | methyl | |
| Time | trimethylhexanoate | valerate | methylbutyrate | ester* | n/iso C5 |
| [min] | (methyl TMH) [%] | [%] | [%] | [%] | ester* [%] |
| 6 | 8 | 49 | 11 | 60 | 81:19 |
| 10 | 10 | 61 | 13 | 74 | 82:18 |
| 30 | 26 | 80 | 18 | 98 | 82:18 |
| 60 | 44 | 81 | 18 | 99 | 82:18 |
| 180 | 70 | 81 | 19 | 100 | 81:19 |
| 720 | 93 | 81 | 19 | 100 | 81:19 |

*sum of possible C5 esters based on the input material

### Example 2—Reaction of Diisobutene and Trans-2-Butene

The experiment in example 2 was performed according to the experimental procedure described in example 1. The only difference was the use of trans-2-butene instead of 1-butene. The results are shown in table 2:

TABLE 2
Progress of reaction according to example 2

| | | | | Yield of | |
|---|---|---|---|---|---|
| | Yield of methyl | Yield of n- | Yield of methyl | C5 | |
| | 3,5,5- | methyl | 2- | methyl | |
| Time | trimethylhexanoate | valerate | methylbutyrate | ester* | n/iso C5 |
| [min] | (methyl TMH) [%] | [%] | [%] | [%] | ester* [%] |
| 6 | 9 | 35 | 8 | 43 | 81:19 |
| 10 | 12 | 49 | 12 | 61 | 80:20 |
| 30 | 30 | 78 | 19 | 97 | 80:20 |
| 60 | 46 | 80 | 20 | 100 | 80:20 |
| 180 | 70 | 80 | 20 | 100 | 80:20 |
| 720 | 92 | 80 | 20 | 100 | 80:20 |

*sum of possible C5 esters based on the input material

### Example 3—Reaction of Diisobutene and Isobutene

The experiment in example 3 was performed according to the experimental procedure described in example 1. The only difference was the use of isobutene instead of 1-butene. The results are shown in table 3:

TABLE 3
Progress of reaction according to example 3

| | Yield of methyl 3,5,5- | Yield of methyl 3- |
|---|---|---|
| Time | trimethylhexanoate | methylbutyrate |
| [min] | (methyl TMH) [%] | [%] |
| 6 | 13 | 46 |
| 10 | 17 | 62 |
| 30 | 32 | 92 |
| 60 | 50 | 100 |
| 180 | 71 | 100 |
| 720 | 92 | 100 |

The experiments performed verify that the object of the invention is achieved by a compound according to the invention.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding European application No. 23186733.4, filed Jul. 20, 2023, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Process for alkoxycarbonylation of diisobutene and a C4 to C7 olefin, wherein the process comprises at least the following steps:
a. providing a diisobutene stream containing 2,4,4-trimethylpent-2-ene and 2,4,4-trimethylpent-1-ene and providing an olefin stream containing the C4 to C7 olefin;
b. alkoxycarbonylation of diisobutene and the C4 to C7 olefin with an alcohol and carbon monoxide in the presence of a homogeneous catalyst system comprising at least one metal of group 8 to 10 of the periodic table of the elements or a compound thereof, a phosphorus-containing ligand and an acid in a common reaction zone to obtain a liquid product mixture comprising at least the esters formed by the alkoxycarbonylation, the homogeneous catalyst system, unreacted olefins and unreacted alcohol;
c. removing the homogeneous catalyst system from the liquid product mixture to obtain a crude product mixture comprising at least the esters formed by the alkoxycarbonylation, the unreacted olefins and unreacted alcohols; and
d. distillative processing of the crude product mixture in at least one distillation column to remove the unreacted alcohols and the unreacted olefins to obtain an ester mixture containing the esters formed.

2. Process according to claim 1, wherein the alkoxycarbonylation in step b is performed at a temperature of 60° C. to 120° C., preferably 65° C. to 110° C., particularly preferably 70° C. to 100° C.

3. Process according to claim 1, wherein the alkoxycarbonylation in step b is performed at a carbon monoxide pressure of 10 to 35 bar, preferably 12.5 to 30 bar, particularly preferably 15 to 25 bar.

4. Process according to claim 1, wherein the content of the metal of group 8 to 10 of the periodic table of the elements, in particular of palladium, in the alkoxycarbonylation reaction solution in step b is 100 to 500 ppm, preferably 150 to 450 ppm, particularly preferably 180 to 350 ppm.

5. Process according to claim 1, wherein the alcohol is a monohydric alcohol having 1 to 4 carbon atoms, especially methanol, ethanol, propanol or butanol.

6. Process according to claim 1, wherein the proportion of 2,4,4-trimethylpent-1-ene in the diisobutene stream is at least 60 mol %, preferably at least 70 mol %.

7. Process according to claim 1, wherein the removal of the homogeneous catalyst system in step c is effected by membrane separation.

8. Process according to claim 7, wherein the homogeneous catalyst system accumulates in the retentate.

9. Process according to claim 1, wherein the diisobutene stream, the olefin stream, the alcohol and the homogeneous catalyst system are initially mixed in a mixing vessel before they are passed into the reaction zone.

10. Process according to claim 1, wherein the distillative processing in step d is carried out in a single distillation column.

11. Process according to claim 10, wherein the pressure in the distillation column is in the range from 0.3 to 2 bar, preferably in the range from 0.4 to 1 bar, particularly preferably in the range from 0.5 to 0.7 bar.

12. Process according to claim 10, wherein the temperature in the bottom of the distillation column is in a range from 80° C. to 160° C.

13. Process according to claim 10, wherein the temperature at the top of the distillation column is in a range from 30° C. to 80° C.

14. Process according to claim 1, wherein a C4 olefin and methanol are employed, as a result of which the ester mixture obtained is a mixture of methyl 3,5,5-trimethylhexanoate and methyl valerate, methyl 2-methylbutyrate, methyl 3-methylbutyrate or a mixture thereof.

15. Process according to claim 1, wherein in step d unreacted alcohol and unreacted olefins are separated and recycled to the alkoxycarbonylation in step b.
